(19) United States
(12) Patent Application Publication
(10) Pub. No.: US 2024/0242783 A1
(43) Pub. Date: Jul. 18, 2024

(54) Ancestry Finder

(71) Applicant: 23andMe, Inc., Sunnyvale, CA (US)

(72) Inventors: John Michael Macpherson, Santa Ana, CA (US); Brian Thomas Naughton, Mountain View, CA (US); Joanna Louise Mountain, Menlo Park, CA (US)

(21) Appl. No.: 18/198,558

(22) Filed: Aug. 7, 2023

Related U.S. Application Data
(63) Continuation of application No. 17/880,566, filed on Aug. 3, 2022, now abandoned.
Application No. 17/880,566 is a continuation of application No. 17/077,930, filed on Oct. 22, 2020, now Pat. No. 11,468,971.
Application No. 17/077,930 is a continuation of application No. 15/664,619, filed on Jul. 31, 2017, now Pat. No. 10,854,318.
Application No. 15/664,619 is a continuation of application No. 12/774,546, filed on May 5, 2010, now abandoned.
Application No. 12/774,546 is a continuation-in-part of application No. 12/644,791, filed on Dec. 22, 2009, now Pat. No. 8,463,554.
(60) Provisional application No. 61/204,195, filed on Dec. 31, 2008.

(51) Int. Cl. G16B 50/30; G06F 16/2457; G06F 16/9535; G06N 5/048; G16B 10/00; G16B 30/00; G16B 50/00

(52) U.S. Cl. CPC G16B 50/30; G06F 16/2457; G06F 16/9535; G06N 5/048; G16B 10/00; G16B 30/00; G16B 50/00

(57) ABSTRACT

Inferring a characteristic of an individual is disclosed. An indication that a first user and a second user have at least one shared chromosomal segment is received. Information about the second user is obtained. A characteristic of the first user is inferred based at least in part on the information about the second user.

### CROSS-REFERENCE TO RELATED APPLICATIONS

This application is a continuation of and claims priority to U.S. patent application Ser. No. 17/880,566, filed Aug. 3, 2022, which is hereby incorporated by reference in its entirety.

U.S. patent application Ser. No. 17/880,566 is a continuation of and claims priority to U.S. patent application Ser. No. 17/077,930, filed Oct. 22, 2020, which is hereby incorporated by reference in its entirety.

U.S. patent application Ser. No. 17/077,930 is a continuation of and claims priority to U.S. patent application Ser. No. 15/664,619, filed Jul. 31, 2017, which is hereby incorporated by reference in its entirety.

U.S. patent application Ser. No. 15/664,619 is a continuation of and claims priority to U.S. patent application Ser. No. 12/774,546, filed May 5, 2010, which is hereby incorporated by reference in its entirety.

U.S. patent application Ser. No. 12/774,546 is a continuation-in-part of and claims priority to U.S. patent application Ser. No. 12/644,791, filed Dec. 22, 2009, which is hereby incorporated by reference in its entirety.

U.S. patent application Ser. No. 12/644,791 claims priority to U.S. provisional patent application No. 61/204,195, filed Dec. 31, 2008, which is hereby incorporated by reference in its entirety.

### BACKGROUND OF THE INVENTION

Genealogy is the study of the history of families and the line of descent from ancestors. It is an interesting subject studied by many professionals as well as hobbyists. Traditional genealogical study techniques typically involve constructing family trees based on surnames and historical records. As gene sequencing technology becomes more accessible, there has been growing interest in genetic ancestry testing in recent years.

Existing genetic ancestry testing techniques are typically based on deoxyribonucleic acid (DNA) information of the Y chromosome (Y-DNA) or DNA information of the mitochondria (mtDNA). Aside from a small amount of mutation, the Y-DNA is passed down unchanged from father to son and therefore is useful for testing patrilineal ancestry of a man. The mtDNA is passed down mostly unchanged from mother to children and therefore is useful for testing a person's matrilineal ancestry. These techniques are found to be effective for identifying individuals that are related many generations ago (e.g., 10 generations or more), but are typically less effective for identifying closer relationships. Further, many relationships that are not strictly patrilineal or matrilineal cannot be easily detected by the existing techniques. In addition, improved techniques for inferring ancestry information for an individual would be desirable.

### BRIEF DESCRIPTION OF THE DRAWINGS

The patent or application file contains at least one drawing executed in color. Copies of this patent or patent application publication with color drawing(s) will be provided by the Patent and Trademark Office upon request and payment of the necessary fee.

Various embodiments of the invention are disclosed in the following detailed description and the accompanying drawings.

FIG. 1 is a block diagram illustrating an embodiment of a relative finding system.

FIG. 2 is a flowchart illustrating an embodiment of a process for finding relatives in a relative finding system.

FIG. 3 is a flowchart illustrating an embodiment of a process for connecting a user with potential relatives found in the database.

FIGS. 4A-4I are screenshots illustrating user interface examples in connection with process 300.

FIG. 5 is a diagram illustrating an embodiment of a process for determining the expected degree of relationship between two users.

FIG. 6 is a diagram illustrating example DNA data used for IBD identification by process 500.

FIG. 7 is a diagram illustrating example simulated relationship distribution patterns for different population groups according to one embodiment.

FIG. 8 is a diagram illustrating an embodiment of a highly parallel IBD identification process.

FIG. 9 is a diagram illustrating an example in which phased data are compared to identify IBD.

FIG. 10 is a block diagram illustrating an embodiment of an ancestry finder system.

FIG. 11 is a flowchart illustrating an embodiment of a process for inferring a characteristic of an individual.

FIG. 12 is a flowchart illustrating an embodiment of a process for determining that a first user and a second user share at least one IBD segment.

FIG. 13 shows an interface example for a table view of an ancestry finder system.

FIG. 14 shows an interface example for the discovery view of a relative finder system that incorporates an ancestry finder system.

FIG. 15 shows an interface example for a karyotype view of an ancestry finder system.

FIG. 16 shows an example of the effect of varying some of the settings in a karyotype view of an ancestry finder system.

FIG. 17 shows an interface example of a karyotype view of an ancestry finder system in which “Number of grandparents from the same country” is 2.

FIG. 18 shows an interface example for a geographical map view of an ancestry finder system.

FIG. 19 is a flowchart illustrating an embodiment of a process for inferring a characteristic of an individual.

### DETAILED DESCRIPTION

The invention can be implemented in numerous ways, including as a process; an apparatus; a system; a composition of matter; a computer program product embodied on a computer readable storage medium; and/or a processor, such as a processor configured to execute instructions stored on and/or provided by a memory coupled to the processor. In this specification, these implementations, or any other form that the invention may take, may be referred to as techniques. In general, the order of the steps of disclosed processes may be altered within the scope of the invention. Unless stated otherwise, a component such as a processor or a memory described as being configured to perform a task may be implemented as a general component that is temporarily configured to perform the task at a given time or a specific component that is manufactured to perform the task. As used herein, the term ‘processor’ refers to one or more devices, circuits, and/or processing cores configured to process data, such as computer program instructions.

A detailed description of one or more embodiments of the invention is provided below along with accompanying figures that illustrate the principles of the invention. The invention is described in connection with such embodiments, but the invention is not limited to any embodiment. The scope of the invention is limited only by the claims and the invention encompasses numerous alternatives, modifications and equivalents. Numerous specific details are set forth in the following description in order to provide a thorough understanding of the invention. These details are provided for the purpose of example and the invention may be practiced according to the claims without some or all of these specific details. For the purpose of clarity, technical material that is known in the technical fields related to the invention has not been described in detail so that the invention is not unnecessarily obscured.

Because of recombination and independent assortment of chromosomes, the autosomal DNA and X chromosome DNA (collectively referred to as recombining DNA) from the parents is shuffled at the next generation, with small amounts of mutation. Thus, only relatives will share long stretches of genome regions where their recombining DNA is completely or nearly identical. Such regions are referred to as “Identical by Descent” (IBD) regions because they arose from the same DNA sequences in an earlier generation. The relative finder technique described below is based at least in part on locating IBD regions in the recombining chromosomes of individuals.

In some embodiments, locating IBD regions includes sequencing the entire genomes of the individuals and comparing the genome sequences. In some embodiments, locating IBD regions includes assaying a large number of markers that tend to vary in different individuals and comparing the markers. Examples of such markers include Single Nucleotide Polymorphisms (SNPs), which are points along the genome with two or more common variations; Short Tandem Repeats (STRs), which are repeated patterns of two or more repeated nucleotide sequences adjacent to each other; and Copy-Number Variants (CNVs), which include longer sequences of DNA that could be present in varying numbers in different individuals. Long stretches of DNA sequences from different individuals' genomes in which markers in the same locations are the same or at least compatible indicate that the rest of the sequences, although not assayed directly, are also likely identical.

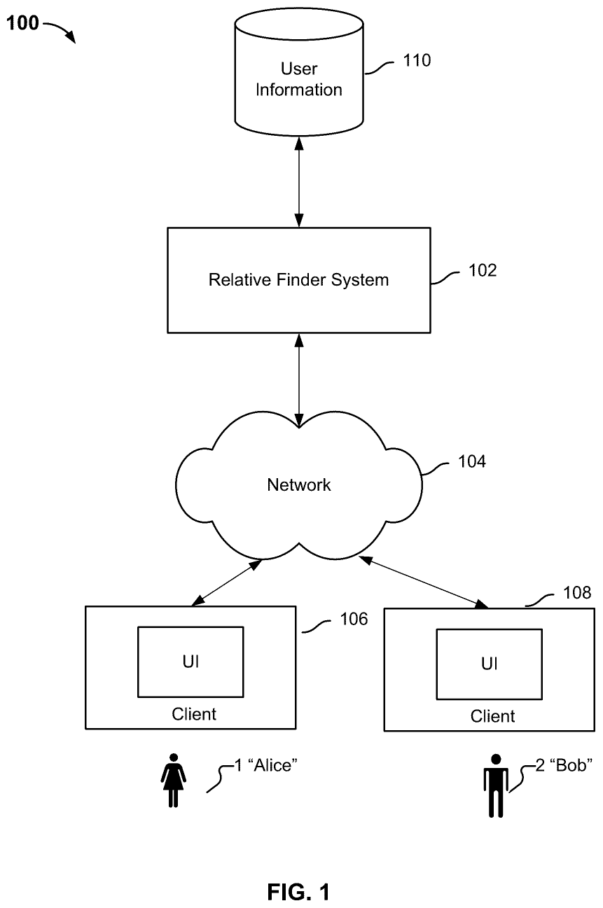

FIG. 1 is a block diagram illustrating an embodiment of a relative finding system. In this example, relative finder system 102 may be implemented using one or more server computers having one or more processors, one or more special purpose computing appliances, or any other appropriate hardware, software, or combinations thereof. The operations of the relative finder system are described in greater detail below. In this example, various users of the system (e.g., user 1 (“Alice”) and user 2 (“Bob”)) access the relative finder system via a network 104 using client devices such as 106 and 108. User information (including genetic information and optionally other personal information such as family information, population group, etc.) pertaining to the users is stored in a database 110, which can be implemented on an integral storage component of the relative finder system, an attached storage device, a separate storage device accessible by the relative finder system, or a combination thereof. Many different arrangements of the physical components are possible in various embodiments. In various embodiments, the entire genome sequences or assayed DNA markers (SNPs, STRs, CNVs, etc.) are stored in the database to facilitate the relative finding process. For example, approximately 650,000 SNPs per individual's genome are assayed and stored in the database in some implementations.

System 100 shown in this example includes genetic and other additional non-genetic information for many users. By comparing the recombining DNA information to identify IBD regions between various users, the relative finder system can identify users within the database that are relatives. Since more distant relationships (second cousins or further) are often unknown to the users themselves, the system allows the users to “opt-in” and receive notifications about the existence of relative relationships. Users are also presented with the option of connecting with their newly found relatives.

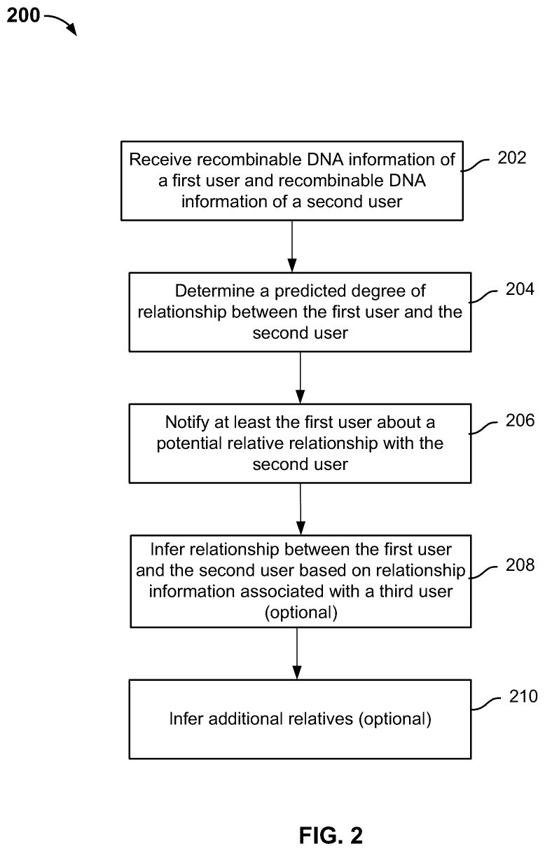

FIG. 2 is a flowchart illustrating an embodiment of a process for finding relatives in a relative finding system. Process 200 may be implemented on a relative finder system such as 100. The process may be invoked, for example, at a user's request to look for potential relatives this user may have in the database or by the system to assess the potential relationships among various users. At 202, recombining DNA information of a first user (e.g., Alice) and of a second user (e.g., Bob) is received. In some embodiments, the information is retrieved from a database that stores recombining DNA information of a plurality of users as well as any additional user information. For purposes of illustration, SNP information is described extensively in this and following examples. Other DNA information such as STR information and/or CNV information may be used in other embodiments.

At 204, a predicted degree of relationship between Alice and Bob is determined. In some embodiments, a range of possible relationships between the users is determined and a prediction of the most likely relationship between the users is made. In some embodiments, it is optionally determined whether the predicted degree of relationship at least meets a threshold. The threshold may be a user configurable value, a system default value, a value configured by the system's operator, or any other appropriate value. For example, Bob may select five generations as the maximum threshold, which means he is interested in discovering relatives with whom the user shares a common ancestor five generations or closer. Alternatively, the system may set a default value minimum of three generations, allowing the users to by default find relatives sharing a common ancestor at least three generations out or beyond. In some embodiments, the system, the user, or both, have the option to set a minimum threshold (e.g., two generations) and a maximum threshold (e.g., six generations) so that the user would discover relatives within a maximum number of generations, but would not be surprised by the discovery of a close relative such as a sibling who was previously unknown to the user.

At 206, Alice or Bob (or both) is notified about her/his relative relationship with the other user. In some embodiments, the system actively notifies the users by sending messages or alerts about the relationship information when it becomes available. Other notification techniques are possible, for example by displaying a list or table of users that are found to be related to the user. Depending on system settings, the potential relatives may be shown anonymously for privacy protection, or shown with visible identities to facilitate making connections. In embodiments where a threshold is set, the user is only notified if the predicted degree of relationship at least meets the threshold. In some embodiments, a user is only notified if both of the user and the potential relative have “opted in” to receive the notification. In various embodiments, the user is notified about certain personal information of the potential relative, the predicted relationship, the possible range of relationships, the amount of DNA matching, or any other appropriate information.

In some embodiments, at 208, the process optionally infers additional relationships or refines estimates of existing relationships between the users based on other relative relationship information, such as the relative relationship information the users have with a third user. For example, although Alice and Bob are only estimated to be 6^{th} cousins after step 204, if among Alice's relatives in the system, a third cousin, Cathy, is also a sibling of Bob's, then Alice and Bob are deemed to be third cousins because of their relative relationships to Cathy. The relative relationships with the third user may be determined based on genetic information and analysis using a process similar to 200, based on non-genetic information such as family tree supplied by one of the users, or both.

In some embodiments, the relatives of the users in the system are optionally checked to infer additional relatives at 210. For example, if Bob is identified as a third cousin of Alice's, then Bob's relatives in the system (such as children, siblings, possibly some of the parents, aunts, uncles, cousins, etc.) are also deemed to be relatives of Alice's. In some embodiments a threshold is applied to limit the relationships within a certain range. Additional notifications about these relatives are optionally generated.

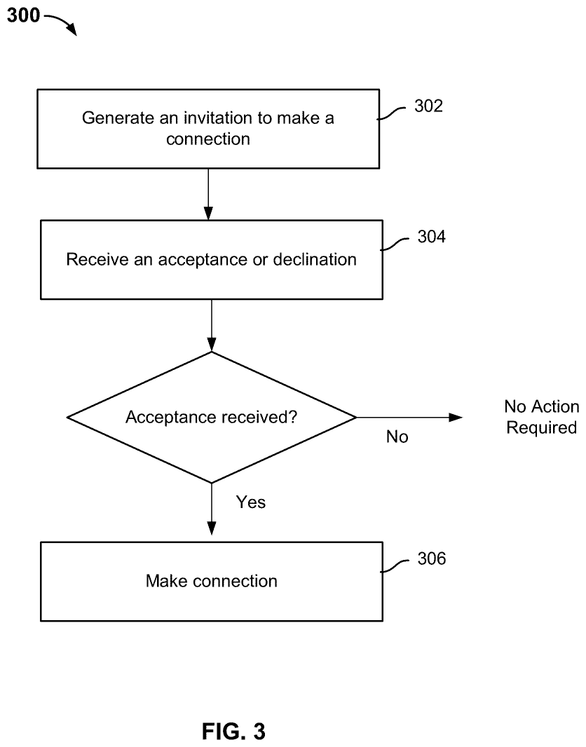

Upon receiving a notification about another user who is a potential relative, the notified user is allowed to make certain choices about how to interact with the potential relative. FIG. 3 is a flowchart illustrating an embodiment of a process for connecting a user with potential relatives found in the database. The process may be implemented on a relative finder system such as 102, a client system such as 106, or a combination thereof. In this example, it is assumed that it has been determined that Alice and Bob are possibly 4th cousins and that Alice has indicated that she would like to be notified about any potential relatives within 6 generations. In this example, process 300 follows 206 of process 200, where a notification is sent to Alice, indicating that a potential relative has been identified. In some embodiments, the identity of Bob is disclosed to Alice. In some embodiments, the identity of Bob is not disclosed initially to protect Bob's privacy.

Upon receiving the notification, Alice decides that she would like to make a connection with the newly found relative. At 302, an invitation from Alice to Bob inviting Bob to make a connection is generated. In various embodiments, the invitation includes information about how Alice and Bob may be related and any personal information Alice wishes to share such as her own ancestry information. Upon receiving the invitation, Bob can accept the invitation or decline. At 304, an acceptance or a declination is received. If a declination is received, no further action is required. In some embodiments, Alice is notified that a declination has been received. If, however, an acceptance is received, at 306, a connection is made between Alice and Bob. In various embodiments, once a connection is made, the identities and any other sharable personal information (e.g., genetic information, family history, phenotype/traits, etc.) of Alice and Bob are revealed to each other and they may interact with each other. In some embodiments, the connection information is updated in the database.

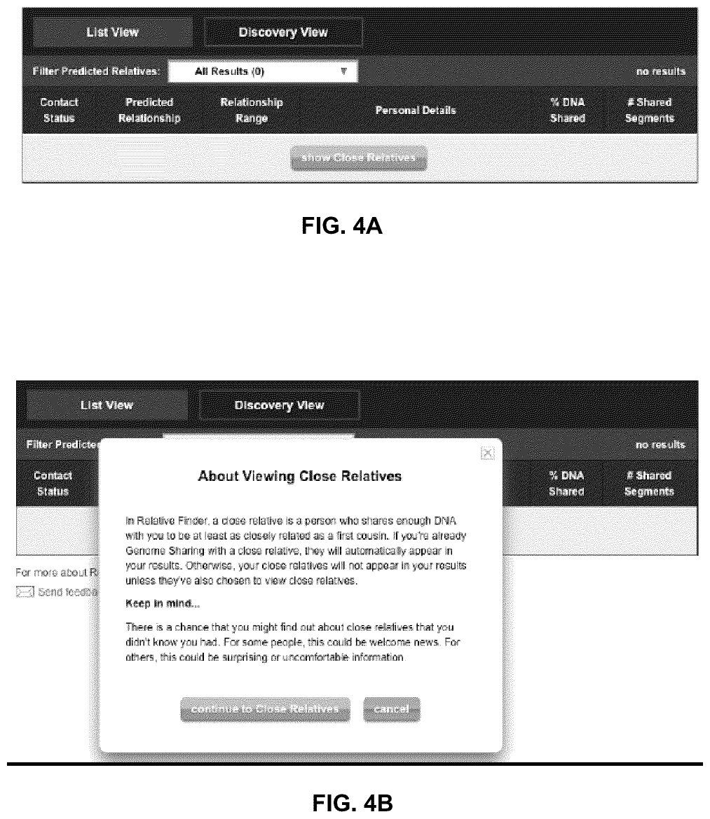

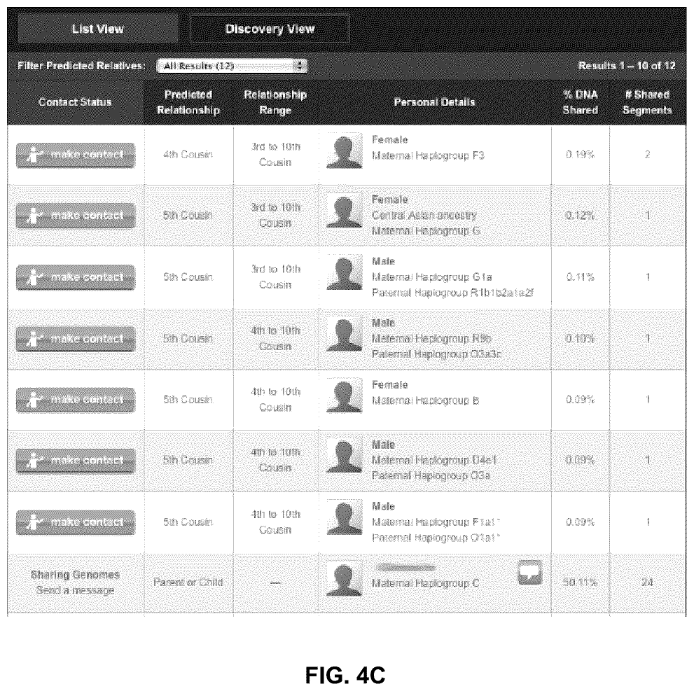

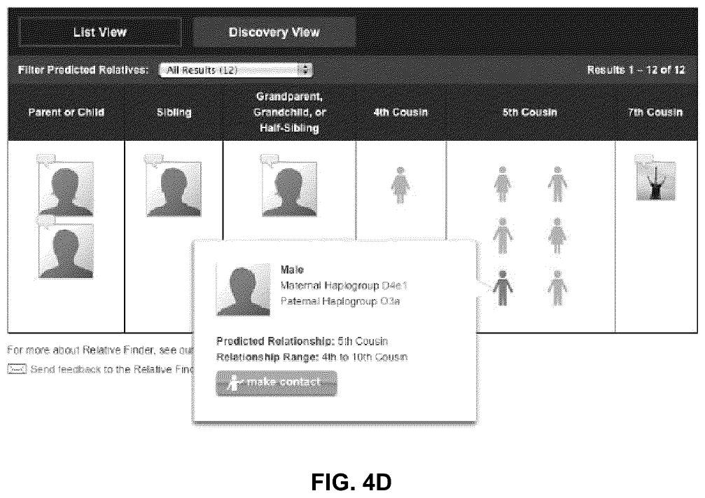

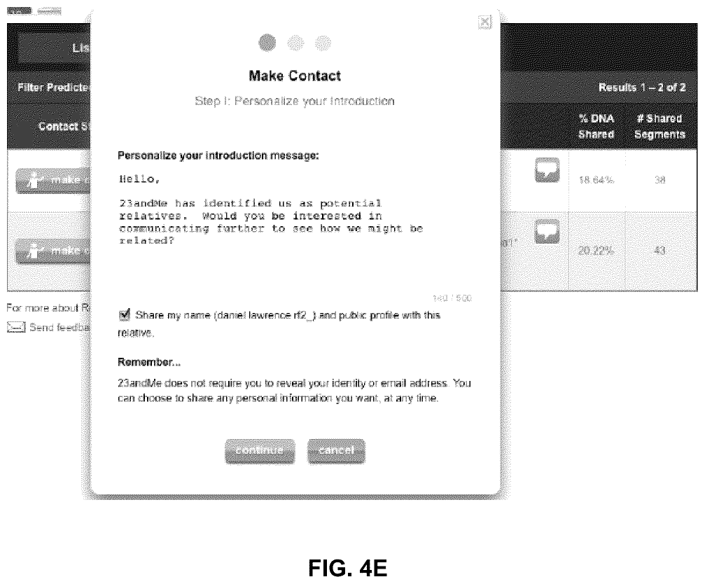

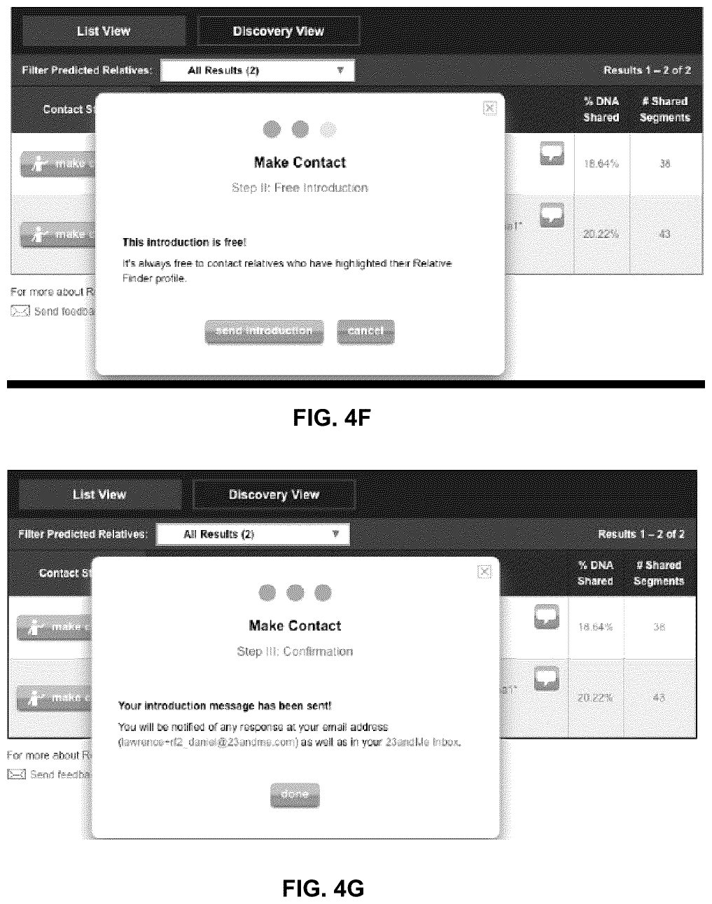

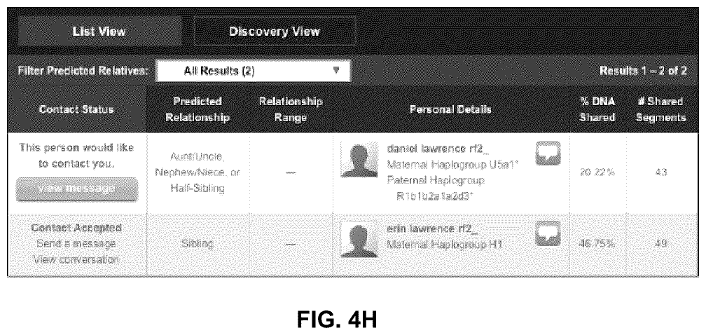

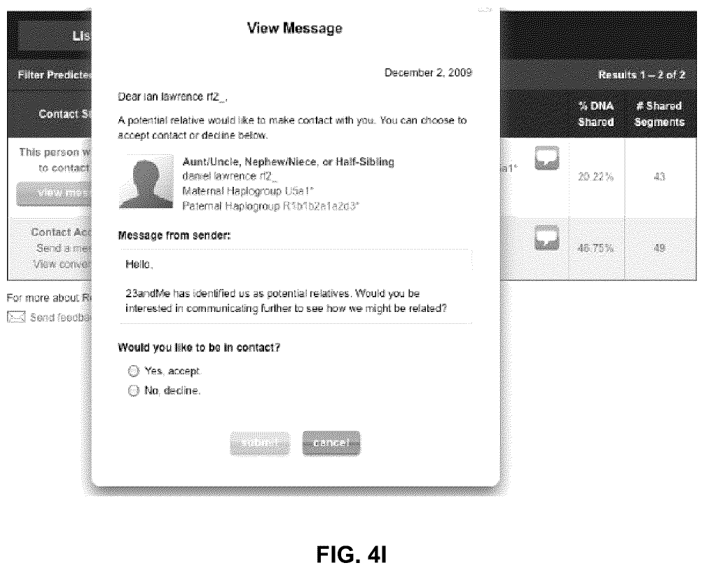

In some embodiments, a user can discover many potential relatives in the database at once. Additional potential relatives are added as more users join the system and make their genetic information available for the relative finding process. FIGS. 4A-4I are screenshots illustrating user interface examples in connection with process 300. In this example, the relative finder application provides two views to the user: the discovery view and the list view.

FIG. 4A shows an interface example for the discovery view at the beginning of the process. No relative has been discovered at this point. In this example, a privacy feature is built into the relative finder application so that close relative information will only be displayed if both the user and the close relative have chosen to view close relatives. This is referred to as the “opt in” feature. The user is further presented with a selection button “show close relatives” to indicate that he/she is interested in finding out about close relatives. FIG. 4B shows a message that is displayed when the user selects “show close relatives”. The message explains to the user how a close relative is defined. In this case, a close relative is defined as a first cousin or closer. In other words, the system has set a default minimum threshold of three degrees. The message further explains that unless there is already an existing connection between the user and the close relative, any newly discovered potential close relatives will not appear in the results unless the potential close relatives have also chosen to view their close relatives. The message further warns about the possibility of finding out about close relatives the user did not know he/she had. The user has the option to proceed with viewing close relatives or cancel the selection.

FIG. 4C shows the results in the discovery view. In this example, seven potential relatives are found in the database. The predicted relationship, the range of possible relationship, certain personal details a potential relative has made public, the amount of DNA a potential relative shares with the user, and the number of DNA segments the potential relative shares with the user are displayed. The user is presented with a “make contact” selection button for each potential relative.

FIG. 4D shows the results in the list view. The potential relatives are sorted according to how close the corresponding predicted relationships are to the user in icon form. The user may select an icon that corresponds to a potential relative and view his/her personal information, the predicted relationship, relationship range, and other additional information. The user can also make contact with the potential relative.

FIGS. 4E-4G show the user interface when the user selects to “make contact” with a potential relative. FIG. 4E shows the first step in making contact, where the user personalizes the introduction message and determine what information the user is willing to share with the potential relative. FIG. 4F shows an optional step in making contact, where the user is told about the cost of using the introduction service. In this case, the introduction is free. FIG. 4G shows the final step, where the introduction message is sent.

FIG. 4H shows the user interface shown to the potential relative upon receiving the introduction message. In this example, the discovery view indicates that a certain user/potential relative has requested to make a contact. The predicted relationship, personal details of the sender, and DNA sharing information are shown to the recipient. The recipient has the option to select “view message” to view the introduction message from the sender.

FIG. 4I shows the message as it is displayed to the recipient. In addition to the content of the message, the recipient is given the option to accept or decline the invitation to be in contact with the sender. If the recipient accepts the invitation, the recipient and the sender become connected and may view each other's information and/or interact with each other.

Many other user interfaces can be used in addition to or as alternatives of the ones shown above. For example, in some embodiments, at least some of the potential relatives are displayed in a family tree.

Determining the relationship between two users in the database is now described. In some embodiments, the determination includes comparing the DNA markers (e.g., SNPs) of two users and identifying IBD regions. The standard SNP based genotyping technology results in genotype calls each having two alleles, one from each half of a chromosome pair. As used herein, a genotype call refers to the identification of the pair of alleles at a particular locus on the chromosome. Genotype calls can be phased or unphased. In phased data, the individual's diploid genotype at a particular locus is resolved into two haplotypes, one for each chromosome. In unphased data, the two alleles are unresolved; in other words, it is uncertain which allele corresponds to which haplotype or chromosome.

The genotype call at a particular SNP location may be a heterozygous call with two different alleles or a homozygous call with two identical alleles. A heterozygous call is represented using two different letters such as AB that correspond to different alleles. Some SNPs are biallelic SNPs with only two possible states for SNPs. Some SNPs have more states, e.g. triallelic. Other representations are possible.

In this example, A is selected to represent an allele with base A and B represents an allele with base G at the SNP location. Other representations are possible. A homozygous call is represented using a pair of identical letters such as AA or BB. The two alleles in a homozygous call are interchangeable because the same allele came from each parent. When two individuals have opposite-homozygous calls at a given SNP location, or, in other words, one person has alleles AA and the other person has alleles BB, it is very likely that the region in which the SNP resides does not have IBD since different alleles came from different ancestors. If, however, the two individuals have compatible calls, that is, both have the same homozygotes (i.e., both people have AA alleles or both have BB alleles), both have heterozygotes (i.e., both people have AB alleles), or one has a heterozygote and the other a homozygote (i.e., one has AB and the other has AA or BB), there is some chance that at least one allele is passed down from the same ancestor and therefore the region in which the SNP resides is IBD. Further, based on statistical computations, if a region has a very low rate of opposite-homozygote occurrence over a substantial distance, it is likely that the individuals inherited the DNA sequence in the region from the same ancestor and the region is therefore deemed to be an IBD region.

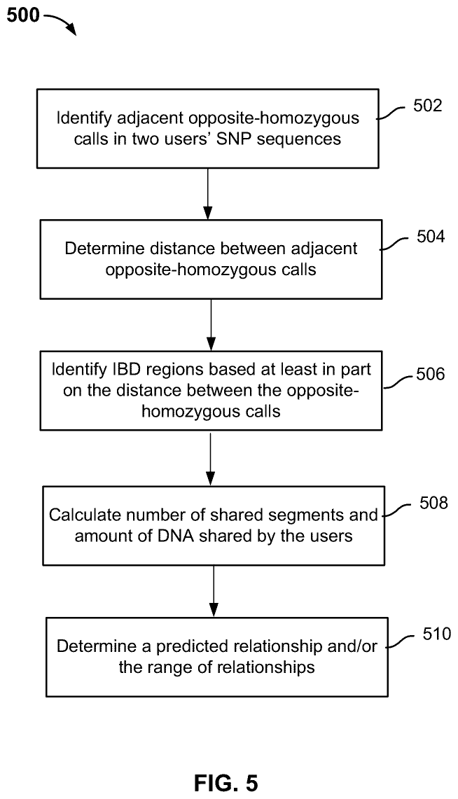

FIG. 5 is a diagram illustrating an embodiment of a process for determining the predicted degree of relationship between two users. Process 500 may be implemented on a relative finder system such as 102 and is applicable to unphased data. At 502, consecutive opposite-homozygous calls in the users' SNPs are identified. The consecutive opposite-homozygous calls can be identified by serially comparing individual SNPs in the users' SNP sequences or in parallel using ffap operations as described below. At 504, the distance between consecutive opposite-homozygous calls is determined. At 506, IBD regions are identified based at least in part on the distance between the opposite-homozygous calls. The distance may be physical distance measured in the number of base pairs or genetic distance accounting for the rate of recombination. For example, in some embodiments, if the genetic distance between the locations of two consecutive opposite-homozygous calls is greater than a threshold of 10 centimorgans (cM), the region between the calls is determined to be an IBD region. This step may be repeated for all the opposite-homozygous calls. A tolerance for genotyping error can be built by allowing some low rate of opposite homozygotes when calculating an IBD segment. In some embodiments, the total number of matching genotype calls is also taken into account when deciding whether the region is IBD. For example, a region may be examined where the distance between consecutive opposite homozygous calls is just below the 10 cM threshold. If a large enough number of genotype calls within that interval match exactly, the interval is deemed IBD.

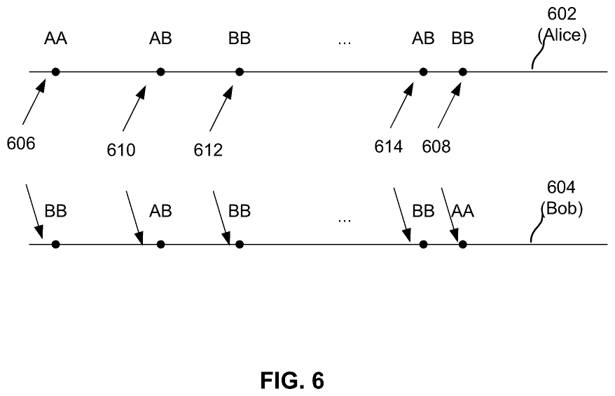

FIG. 6 is a diagram illustrating example DNA data used for IBD identification by process 500. 602 and 604 correspond to the SNP sequences of Alice and Bob, respectively. At location 606, the alleles of Alice and Bob are opposite-homozygotes, suggesting that the SNP at this location resides in a non-IBD region. Similarly, at location 608, the opposite-homozygotes suggest a non-IBD region. At location 610, however, both pairs of alleles are heterozygotes, suggesting that there is potential for IBD. Similarly, there is potential for IBD at location 612, where both pairs of alleles are identical homozygotes, and at location 614, where Alice's pair of alleles is heterozygous and Bob's is homozygous. If there is no other opposite-homozygote between 606 and 608 and there are a large number of compatible calls between the two locations, it is then likely that the region between 606 and 608 is an IBD region.

Returning to FIG. 5, at 508, the number of shared IBD segments and the amount of DNA shared by the two users are computed based on the IBD. In some embodiments, the longest IBD segment is also determined. In some embodiments, the amount of DNA shared includes the sum of the lengths of IBD regions and/or percentage of DNA shared. The sum is referred to as IBD_{half} or half IBD because the individuals share DNA identical by descent for at least one of the homologous chromosomes. At 510, the predicted relationship between the users, the range of possible relationships, or both, is determined using the IBD_{half} and number of segments, based on the distribution pattern of IBD_{half} and shared segments for different types of relationships. For example, in a first degree parent/child relationship, the individuals have IBD_{half} that is 100% the total length of all the autosomal chromosomes and 22 shared autosomal chromosome segments; in a second degree grandparent/grandchild relationship, the individuals have IBD_{half} that is approximately half the total length of all the autosomal chromosomes and many more shared segments; in each subsequent degree of relationship, the percentage of IBD_{half} of the total length is about 50% of the previous degree. Also, for more distant relationships, in each subsequent degree of relationship, the number of shared segments is approximately half of the previous number.

In various embodiments, the effects of genotyping error are accounted for and corrected. In some embodiments, certain genotyped SNPs are removed from consideration if there are a large number of Mendelian errors when comparing data from known parent/offspring trios. In some embodiments, SNPs that have a high no-call rate or otherwise failed quality control measures during the assay process are removed. In some embodiments, in an IBD segment, an occasional opposite-homozygote is allowed if there is sufficient opposite-homozygotes-free distance (e.g., at least 3 cM and 300 SNPs) surrounding the opposite-homozygote.

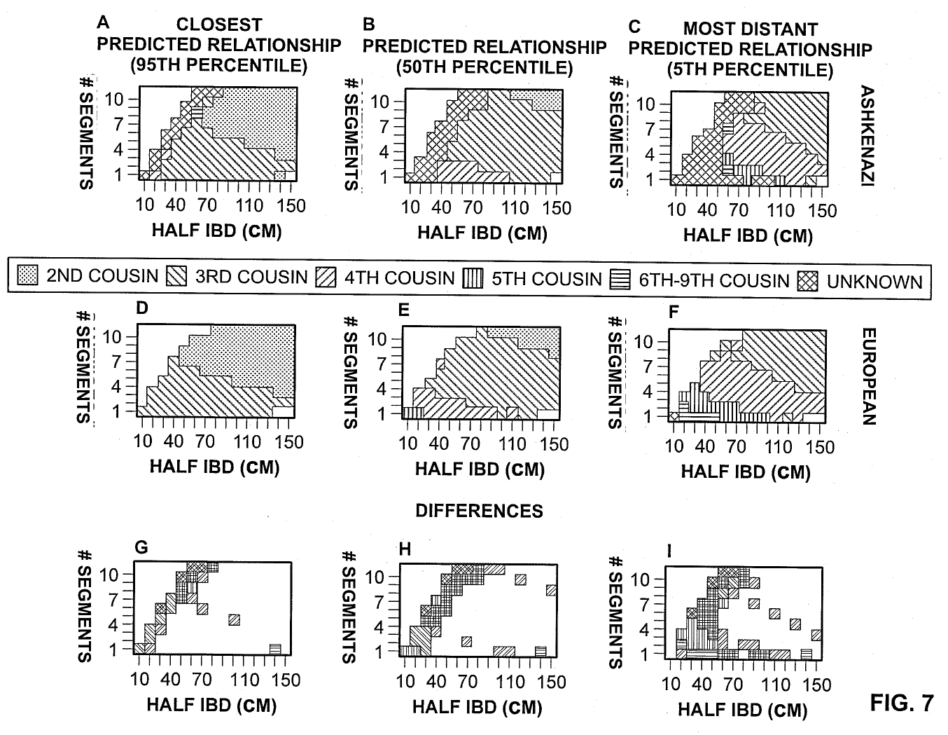

There is a statistical range of possible relationships for the same IBD_{half} and shared segment number. In some embodiments, the distribution patterns are determined empirically based on survey of real populations. Different population groups may exhibit different distribution patterns. For example, the level of homozygosity within endogamous populations is found to be higher than in populations receiving gene flow from other groups. In some embodiments, the bounds of particular relationships are estimated using simulations of IBD using generated family trees. Based at least in part on the distribution patterns, the IBD_{half}, and shared number of segments, the degree of relationship between two individuals can be estimated. FIG. 7 is a diagram illustrating example simulated relationship distribution patterns for different population groups according to one embodiment. In particular, Ashkenazi Jews and Europeans are two population groups surveyed. In panels A-C, for each combination of IBD_{half} and the number of IBD segments in an Ashkenazi sample group, the 95%, 50% and 5% of obtained nth degree cousinships from 1 million simulated pedigrees are plotted. In panels D-F, for each combination of IBD_{half} and the number of IBD segments in a European sample group, the 95%, 50% and 5% of obtained nth degree cousinships from 1 million simulated pedigrees are plotted. In panels G-I, the differences between Ashkenazi and European distant cousinship for the prior panels are represented. Each nth cousinship category is scaled by the expected number of nth degree cousins given a model of population growth. Simulations are conducted by specifying an extended pedigree and creating simulated genomes for the pedigree by simulating the mating of individuals drawn from a pool of empirical genomes. Pairs of individuals who appear to share IBD_{half} that was not inherited through the specified simulated pedigree are marked as “unknown” in panels A-F. Thus, special distribution patterns can be used to find relatives of users who have indicated that they belong to certain distinctive population groups such as the Ashkenazi.

The amount of IBD sharing is used in some embodiments to identify different population groups. For example, for a given degree of relationship, since Ashkenazi tend to have much more IBD sharing than non-Ashkenazi Europeans, users may be classified as either Ashkenazi or non-Ashkenazi Europeans based on the number and pattern of IBD matches.

In some embodiments, instead of, or in addition to, determining the relationship based on the overall number of IBD segments and percent DNA shared, individual chromosomes are examined to determine the relationship. For example, X chromosome information is received in some embodiments in addition to the autosomal chromosomes. The X chromosomes of the users are also processed to identify IBD. Since one of the X chromosomes in a female user is passed on from her father without recombination, the female inherits one X chromosome from her maternal grandmother and another one from her mother. Thus, the X chromosome undergoes recombination at a slower rate compared to autosomal chromosomes and more distant relationships can be predicted using IBD found on the X chromosomes.

In some embodiments, analyses of mutations within IBD segments can be used to estimate ages of the IBD segments and refine estimates of relationships between users.

In some embodiments, the relationship determined is verified using non-DNA information. For example, the relationship may be checked against the users' family tree information, birth records, or other user information.

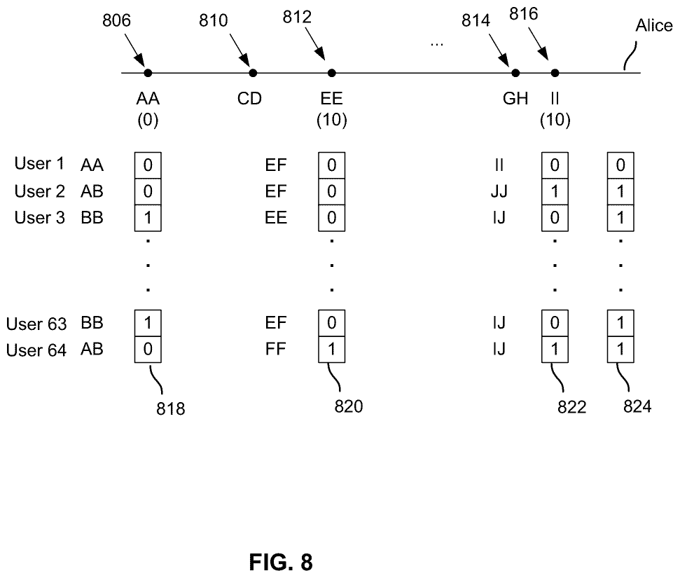

In some embodiments, the efficiency of IBD region identification is improved by comparing a user's DNA information with the DNA information of multiple other users in parallel and using bitwise operations. FIG. 8 is a diagram illustrating an embodiment of a highly parallel IBD identification process. Alice's SNP calls are compared with those of multiple other users. Alice's SNP calls are pre-processed to identify ones that are homozygous. Alice's heterozygous calls are not further processed since they always indicate that there is possibility of IBD with another user. For each SNP call in Alice's genome that is homozygous, the zygosity states in the corresponding SNP calls in the other users are encoded. In this example, compatible calls (e.g., heterozygous calls and same homozygous calls) are encoded as 0 and opposite-homozygous calls are encoded as 1. For example, for homozygous SNP call AA at location 806, opposite-homozygous calls BB are encoded as 1 and compatible calls (AA and AB) are encoded as 0; for homozygous SNP call EE at location 812, opposite-homozygous calls FF are encoded as 1 and compatible calls (EE and EF) are encoded as 0, etc. The encoded representations are stored in arrays such as 818, 820, and 824. In some embodiments, the length of the array is the same as the word length of the processor to achieve greater processing efficiency. For example, in a 64-bit processing system, the array length is set to 64 and the zygosity of 64 users' SNP calls are encoded and stored in the array.

A bitwise operation is performed on the encoded arrays to determine whether a section of DNA such as the section between locations 806 and 810 includes opposite-homozygous calls. In this example, a bitwise OR operation is performed to generate a result array 824. Any user with no opposite-homozygous calls between beginning location 806 and ending location 816 results in an entry value of 0 in array 824. The corresponding DNA segment, therefore, is deemed as an IBD region for such user and Alice. In contrast, users with opposite-homozygotes result in corresponding entry values of 1 in array 824 and they are deemed not to share IBD with Alice in this region. In the example shown, user 1 shares IBD with Alice while other users do not.

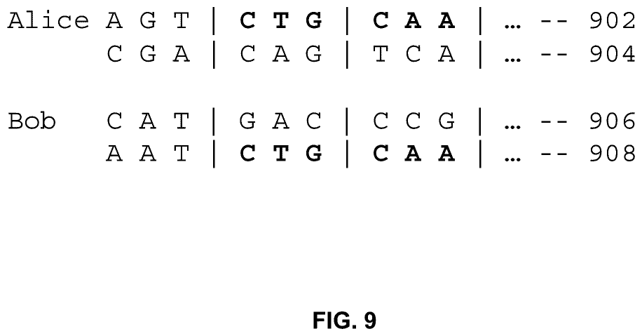

In some embodiments, phased data is used instead of unphased data. These data can come directly from assays that produce phased data, or from statistical processing of unphased data. IBD regions are determined by matching the SNP sequences between users. In some embodiments, sequences of SNPs are stored in dictionaries using a hash-table data structure for the ease of comparison. FIG. 9 is a diagram illustrating an example in which phased data are compared to identify IBD. The sequences are split along pre-defined intervals into non-overlapping words. Other embodiments may use overlapping words. Although a preset length of 3 is used for purposes of illustration in the example shown, many implementations may use words of longer lengths (e.g. 100). Also, the length does not have to be the same for every location. In FIG. 9, in Alice's chromosome pair 1, chromosome 902 is represented by words AGT, CTG, CAA, . . . and chromosome 904 is represented by CGA, CAG, TCA, . . . . At each location, the words are stored in a hash table that includes information about a plurality of users to enable constant retrieval of which users carry matching haplotypes. Similar hash tables are constructed for other sequences starting at other locations. To determine whether Bob's chromosome pair 1 shares any IBD with Alice's, Bob's sequences are processed into words at the same locations as Alice's. Thus, Bob's chromosome 906 yields CAT, GAC, CCG, . . . and chromosome 908 yields AAT, CTG, CAA, . . . Every word from Bob's chromosomes is then looked up in the corresponding hash table to check whether any other users have the same word at that location in their genomes. In the example shown, the second and third words of chromosome 908 match second and third words of Alice's chromosome 902. This indicates that SNP sequence CTGCAA is present in both chromosomes and suggests the possibility of IBD sharing. If enough matching words are present in close proximity to each other, the region would be deemed IBD.

In some embodiments, relative relationships found using the techniques described above are used to infer characteristics about the users that are related to each other. In some embodiments, the inferred characteristic is based on non-genetic information pertaining to the related users. For example, if a user is found to have a number of relatives that belong to a particular population group, then an inference is made that the user may also belong to the same population group. In some embodiments, genetic information is used to infer characteristics, in particular characteristics specific to shared IBD segments of the related users. Assume, for example, that Alice has sequenced her entire genome but her relatives in the system have only genotyped SNP data. If Alice's genome sequence indicates that she may have inherited a disease gene, then, with Alice's permission, Alice's relatives who have shared IBD with Alice in the same region that includes the disease gene may be notified that they are at risk for the same disease.

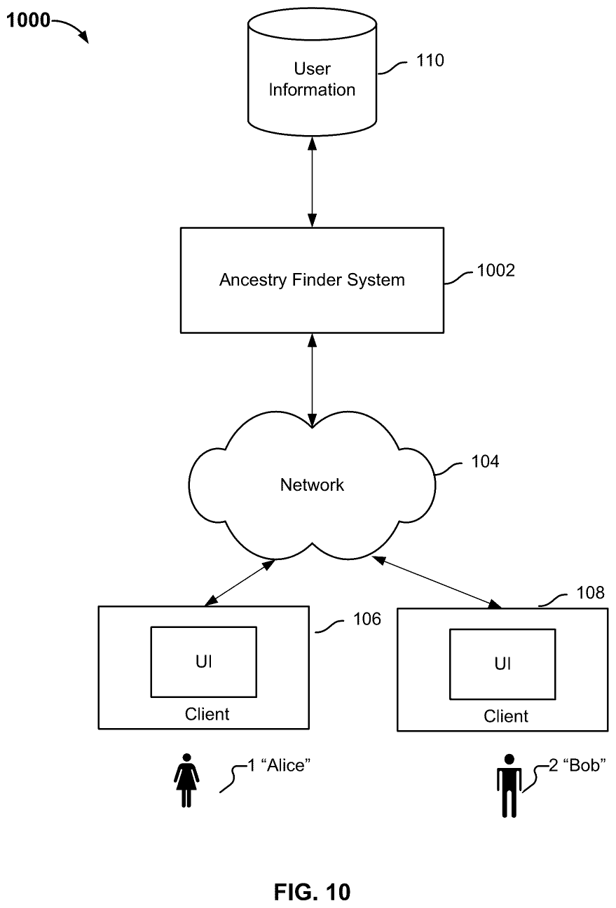

FIG. 10 is a block diagram illustrating an embodiment of an ancestry finder system. In this example, ancestry finder system 102 may be implemented using one or more server computers having one or more processors, one or more special purpose computing appliances, or any other appropriate hardware, software, or combinations thereof. The operations of the ancestry finder system are described in greater detail below. In this example, various users of the system (e.g., user 1 (“Alice”) and user 2 (“Bob”)) access the ancestry finder system via a network 104 using client devices such as 106 and 108. User information (including genetic information and optionally other personal information such as family information, population group, etc.) pertaining to the users is stored in a database 110, which can be implemented on an integral storage component of the ancestry finder system, an attached storage device, a separate storage device accessible by the ancestry finder system, or a combination thereof. Many different arrangements of the physical components are possible in various embodiments. In some embodiments, the entire genome sequences or assayed DNA markers (SNPs, STRs, CNVs, etc.) are stored in the database to facilitate the relative finding process. For example, approximately 650,000 SNPs per individual's genome are assayed and stored in the database in some implementations.

System 1000 shown in this example includes genetic and other additional non-genetic information for many users. By comparing the recombining DNA information to identify shared IBD regions between various users, the ancestry finder system can infer ancestry information or other characteristics of a user.

In some embodiments, database 110 includes results of a survey of users. For example, the survey may be an ancestry survey that requests information such as the place (e.g., country and city) of birth, race, and ethnicity of the user. In some embodiments, the survey also requests information regarding relatives of the user, such as the place of birth and race/ethnicity of each of the user's parents and grandparents (if known to the user). For example, Alice may provide her place of birth and ethnicity, as well as the places of birth and ethnicities of each of her parents and grandparents. Such information may be useful for determining information about the ancestry of other users who are related to Alice and/or have a common IBD segment with Alice, as further described below. In some embodiments, Alice may also provide ancestry information for her direct maternal line (mother's mother's mother's . . . mother) as far back as possible. Alice's brother (or any male) might provide ancestry information for his direct paternal line (father's father's father's . . . father) as far back as possible. In various embodiments, the user survey results are stored separately from database 110, such as in a separate database. In various embodiments, relative finder system 102 and ancestry finder system 1002 are part of the same system and may share data.

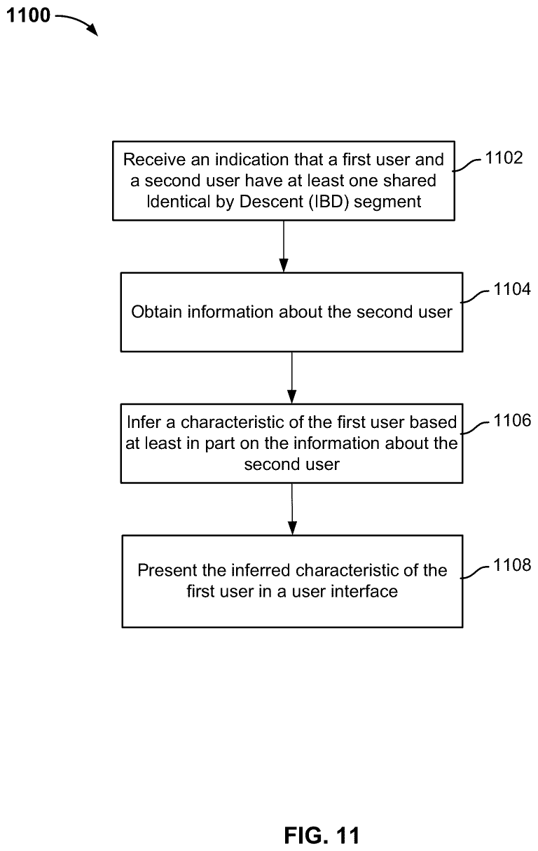

FIG. 11 is a flowchart illustrating an embodiment of a process for inferring a characteristic of an individual. Process 1100 may be implemented on an ancestry finder system such as 1100. In some embodiments, if a user has a chromosomal segment that is associated with a particular population group (e.g., based on other users having that chromosomal segment who have identified themselves as being associated with the population group), this process infers that the user has ancestry associated with that population group. The process may be invoked, for example, at a user's request to look for ancestry information and/or potential relatives this user may have in the database. One or more steps of process 1100 may be part of a batch process used to obtain ancestry information (or information used to infer ancestry information) for a plurality of users.

At 1102, an indication that a first user (e.g., Alice) and a second user (e.g., Bob) have at least one shared IBD segment is received. In some embodiments, it is determined that the first user and the second user have at least one shared IBD segment based on information retrieved from a database that stores recombining DNA information of a plurality of users as well as additional user information. For purposes of illustration, SNP information is described extensively in this and following examples. Other DNA information such as STR information, CNV information, exomic sequence information, and/or full sequence information may be used in other embodiments. In some embodiments, the second user is an individual from a reference database, and not necessarily a user of the ancestry finder system.

At 1104, information about the second user is obtained. In some embodiments, the information about the second user comprises one or more characteristics of one or more relatives of the second user. In some embodiments, at least one characteristic is ancestry information. For example, the information about the second user could comprise ancestry information about the four grandparents of the second user, such as the birthplaces of the four grandparents. Such information could, for example, have been provided by the second user when filling out a survey of characteristics of the user and the user's relatives, such as an ancestry survey, an example of which was previously described. In other embodiments, such information could be provided by a reference database. In some embodiments, the information about the second user is obtained from a database, such as database 110.

At 1106, a characteristic of the first user is inferred based at least in part on the information about the second user. If the first user and second user share at least one common IBD segment, information about the first user could be inferred from known information about the second user. For example, if it is known that the second user's four grandparents were all born in Ireland, than it can be inferred that the first user has at least some Irish ancestry, or at least that there is someone of Irish ancestry associated with that segment value. Characteristics can include ancestry information. Other examples of characteristics that can be inferred besides ancestry information include any other inherited characteristic, such as information associated with diseases, traits, or any other form of phenotypic information.

At 1108, the inferred characteristic of the first user is presented in a user interface. Various examples of user interfaces for presenting, displaying, or notifying of the inferred characteristic are possible. Some examples of user interfaces include a table view, an (extended) discovery view, a karyotype view, a geographical map view, as more fully described below. For example, a discovery view may display a list or table of users that are found to be related to the user based on the relative finder system. In addition to displaying relative finder results, ancestry information is displayed next to each relative. For example, next to each relative, there may be a column for each grandparent of that relative. An indication of ancestry information for each grandparent may be displayed in each column, as more fully illustrated below. Other examples of user interfaces include a family tree, which displays a family tree filled with inferred ancestry information about each relative. In some embodiments, the system actively notifies the users by sending messages or alerts about ancestry information when it becomes available or at a given interval.

In some embodiments, it is determined that for a particular segment, the first user shares IBD with other users besides the second user. If the other users that share this chromosomal segment have conflicting inferred ancestry (or other characteristic) information, then this conflict may be handled in various ways in various embodiments. In some embodiments, all the information is presented. In some embodiments, the information is processed to infer ancestry (or other characteristic information). For example, the information may be processed using a majority rules technique. For example, if the ancestry associated with the majority of the matching users is German, then it is inferred that that segment is associated with German ancestry. In some embodiments, for each segment and each possible value of each segment, the information is processed (e.g., using majority rules), and the processed result is pre-computed and stored for future reference by any of the matching users.

In some embodiments, 1102 is part of a batch process in which a plurality of users (e.g., from a database) are preprocessed to determine shared IBD segments. The results comprise shared IBD segments for a plurality of users. For example, for a particular user, there may be 278 matches to various segments of the user's chromosomes. At 1104, these results are cross-referenced with a database of information (e.g., ancestry information) about those matching users. For example, of the 278 matches, 49 of those users have completed an ancestry information survey that can be used to infer ancestry information about the particular user.

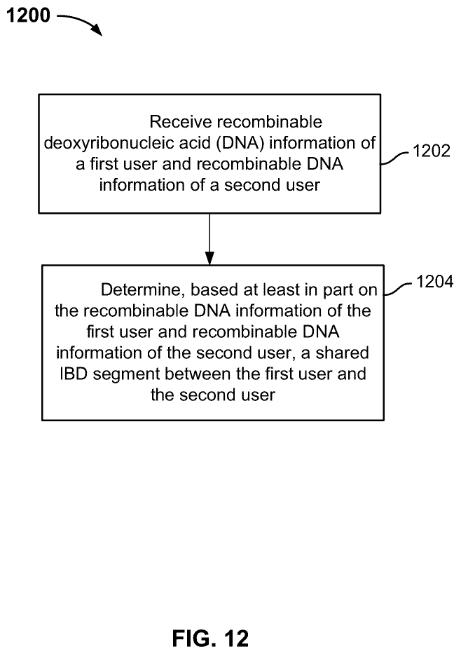

FIG. 12 is a flowchart illustrating an embodiment of a process for determining that a first user and a second user share at least one IBD segment. Process 1200 may be implemented on an ancestry finder system such as 1100. The process may be performed, for example, at 1102 of process 1100. At 1202, recombining DNA information of a first user and recombining DNA information of a second user are received. At 1204, a shared IBD segment between the first user and the second user is determined based at least in part on the recombining DNA information of the first user and recombining DNA information of the second user. Any appropriate technique may be used to determine the shared IBD segment. For example, in some embodiments, one or more steps of process 500 are used to determine the shared IBD segment. In some embodiments, one or more steps of the process described with respect to FIG. 6, 8, or 9 is used to determine the shared IBD segment.

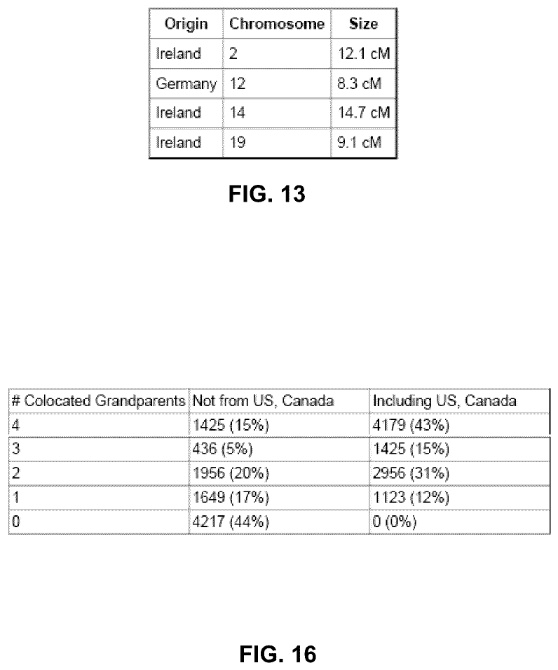

FIG. 13 shows an interface example for a table view of an ancestry finder system. In some embodiments, this interface is used to present inferred ancestry information for a user at 1108. In this example, a table is displayed or presented that illustrates the results a typical user of European ancestry might receive. Each row in the table corresponds to a chromosomal segment that the user shares by IBD with another individual (i.e., a relative finder hit). Three of the segments are to (one or more) individuals of Irish ancestry, and one is to an individual of German ancestry. This table shows a relatively low number of ancestry finder hits. In other words, only about 40 cM (roughtly 40 Mb), or a bit more than half a percent of this user's genome. (The calculation is 40 cM/6000 cM=0.7%. The denominator is not 3000 cM, the length of the diploid genome, but twice that, the length of the haploid genome, because there could be an ancestry finder hit to either chromosome. This typically is the case with Ashkenazim.) How much of the genome is covered depends on the number of individuals in the database from the same subpopulations as the user and the consanguinity of that subpopulation. A hit or match means that at least a portion of the user's genome was found in the indicated part of the world prior to the era of intercontinental travel.

In various embodiments, other types of interfaces, tables, charts, or views may be used. For example, a table of the top ten countries associated with the user's ancestral origin may be presented or displayed. These top ten countries are determined based on the total number of non-overlapping DNA segments attributable to each country. A pie chart may be used to show the breakdown among countries.

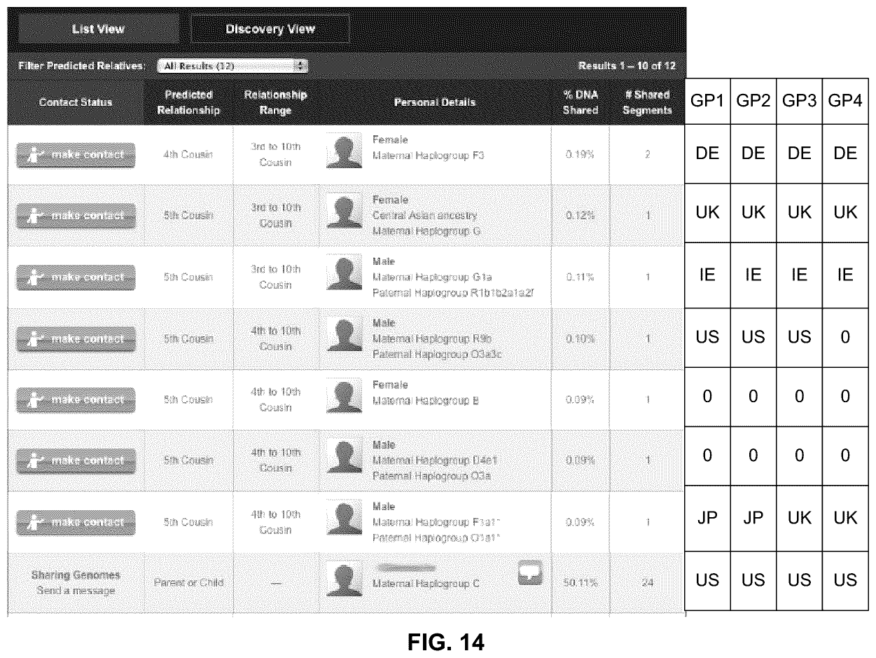

FIG. 14 shows an interface example for the discovery view of a relative finder system that incorporates an ancestry finder system. In some embodiments, this interface is used to present inferred ancestry information for a user at 1108. In this example, the user interface of FIG. 4C is shown in addition to four new columns: GP1-GP4, representing Grandparents 1-4. For each relative, ancestry information is shown in each of the columns for that relative. In this example, the ancestry information is represented by codes that indicate birth countries of the grandparents. DE is Germany, UK is the United Kingdom, IE is Ireland, US is the United States, and JP is Japan. For example, the first row shows a 4^{th} cousin with four grandparents who are all of German ancestry. In other embodiments, other ancestry information could be displayed, such as race/ethnicity (e.g., NA for Native American). In various embodiments, various other ancestry information could be provided, such as information about the relative's parents or siblings. In various embodiments, various other information about characteristics of the relative could be provided, including disease, trait, or any other form of phenotypic information

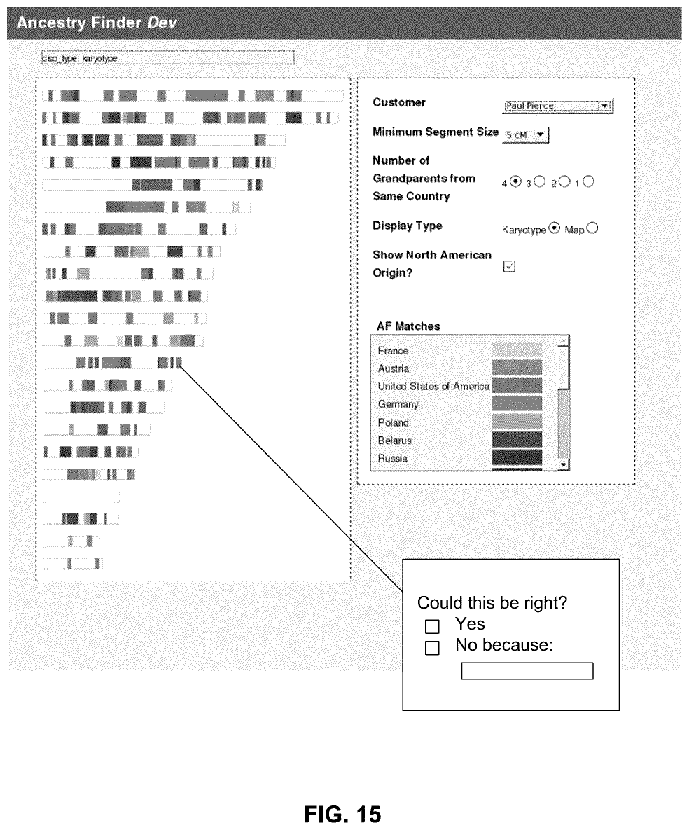

FIG. 15 shows an interface example for a karyotype view of an ancestry finder system. In some embodiments, this interface is used to present inferred ancestry information for a user “Paul Pierce” at 1108.

In this example, the user interface shows a graphic of a user's chromosome on the left hand side. On each chromosome, various segments are colored based on with which ancestry (France, Austria, United States, etc.) that segment is associated. In some embodiments, a segment is associated with an ancestry based on an IBD match with an individual of known ancestry. For example, if a user's segment has an IBD match with another individual, and the birth country of all four grandparents of that individual is provided or known, then it can be inferred that the user's segment is associated with that birth country. In other words, that shared IBD segment was associated with that part of the world (prior to the era of intercontinental travel).

On the right hand side, a control panel is shown. In the control panel, the customer (user) name is shown, along with user configurable settings. “Minimum segment size” is the minimum length of a matching IBD segment, in this example, 5 cM. Minimum segment size governs the minimum length of displayed ancestry finder hits. It's given in centiMorgans (cM) in the example shown, but can also be expressed in terms of base pairs, kilobases, megabases, or any other appropriate unit. In this example, most hits are short, so increasing the minimum quickly reduces the number of segments displayed. These longer hits, although fewer, are increasingly likely to indicate recent shared ancestry, and therefore to represent genuine/applicable ancestry finder results. In this example, the great majority of IBD hits, and thus ancestry finder segments, are between 5 cM and 10 cM in length. The lower end is controlled by a few threshold parameters in the IBD matching technique and can take any appropriate value. The lower edge might be different for the IBD hits on live. One range might be 5 cm to 20 cM (20 cM is the approximate length of chromosome 22). Although a pull down menu is shown, in various embodiments, a slider or other element could be used, with a default initial value.

“Number of grandparents from the same country” indicates the minimum number of grandparents from the same country in order for an association to be made, as will be more fully described below. In this example, ancestry associations for a segment are only made if the birth countries of all four grandparents are the same. “Display type” indicates that this is a karyotype display.

“Show North American origin?” indicates whether to include the United States and Canada as ancestry finder matches. For example, a user may desire to exclude North American origin matches because they may not be as informative since most individuals born in North America have ancestry in other parts of the world. In some cases, if this option is unchecked, then relatively fewer number of hits will result because there are many users whose four grandparents are from the United States or Canada. This is a toggle in the example, but other user interface elements may be used.

In some embodiments, a dialog box such as the one shown may be used for a user to provide feedback in this view or any other view (such as the discovery view). For example, the dialog box may open when a cursor hovers over one of the segments. The shown dialog box asks the user whether the indicated ancestry information could be right. The user can indicate “Yes” or “No” and provide a reason. In some embodiments, this feedback is used by the ancestry finder system to improve future inferences. For example, a user may see one segment that is colored brown to indicate Japan. If the user knows that he does not have any Japanese ancestry, he may select “No” in the dialog box. This may happen, for example, if the individual with the matching IBD segment to the user has indicated his grandparents are all from Japan due to misinformation. This could also happen if there is misleading information. For example a user could be born in Australia, but not be Aborigines. In some cases, a particular IBD segment may be found in more than one part of the world, leading to this result. For example, there may be tracks of chromosome that do not actually correspond to recent shared ancestry—they could correspond to old ancestry and be shared by many users.

In various embodiments, other user configurable settings may be included, such as an option to Show Public Reference Individuals and/or Show Relative Finder Users. There may be hits to customers via the ancestry survey, and to anonymous reference individuals of known ancestry from public genotype databases. The customer may wish to toggle visibility of the public individuals/Relative Finder. In some embodiments, this could rotate through three states: Both->Relative Finder Users Only->Public Only.

An option to “Notify Me of Changes to My Ancestry Finder Results” may be provided. Ancestry finder results will change/improve with time. This would allow a user to indicate whether and how the user wishes to be notified of these changes. For example, the user could request to be notified as soon as they happen (up to daily), weekly in a batch, monthly in a batch. The user could indicate which results the user wants to be notified of (e.g., four grandparents, three grandparents, and/or Non-North American).

FIG. 16 shows an example of the effect of varying some of the settings in a karyotype view of an ancestry finder system, such as that shown in FIG. 15.

“Number of Grandparents from Same Country” is the minimum number of co-located grandparents for an ancestry finder match to be made. This governs how many grandparents of the individual matching a user must be associated with the same country/ethnicity. For example, if four, then only segments for which all four grandparents come from the same country will be shown. The table shows the overall distribution of ancestry survey responses across an example database of users. The possible values are integers from four to one, inclusive.

Varying “Number of Grandparents from Same Country” and “Show North American Origin?” affects the number and informativeness of ancestry finder hits displayed. For example, in an example database, most customers of European descent have about 100 relative finder hits in the database, and about a third of the users have taken an ancestry survey, this means there are 100*(⅓) or about 30 users who match and have provided ancestry information via the ancestry survey. Generally speaking, the most informative ancestry finder matches are those when there is a match who has four grandparents from the same country, and that country is not the US or Canada. Since such matches are relatively rare in the example database, perhaps 0 to 5 of the approximately 30 matches from above, users may wish to see their other matches, even though they may be less informative. Decreasing the number of grandparents required to be from the same country from 4 to 3 to 2 to 1 increases the number of hits displayed, as does allowing matches from the US and Canada to be shown.

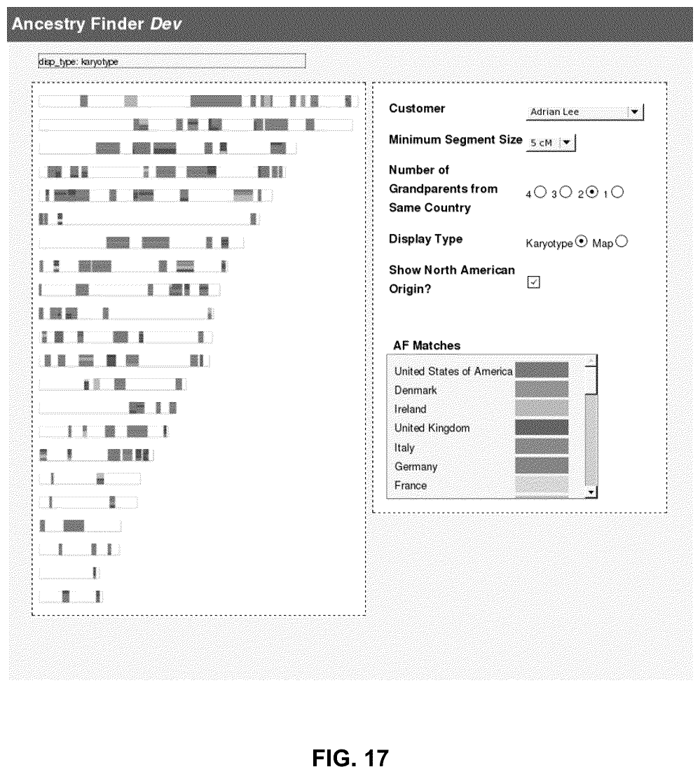

FIG. 17 shows an interface example of a karyotype view of an ancestry finder system in which “Number of grandparents from the same country” is 2. In this example, the interface of FIG. 15 is shown for a user “Adrian Lee” who has selected 2 as the “Number of grandparents from the same country.” This means that for each shared IBD segment shown (colored), the user had shared IBD with someone who has at least two grandparents from the same country. As shown, some of the segments show two or more colors (split horizontally) to indicate that that segment is IBD matched with someone whose grandparents could be from as many as three different countries. In contrast, FIG. 15 shows only one color per segment because all four grandparents must be from the same country. In various embodiments, a variety of graphical elements or visual cues may be used to depict such inferences about characteristics (such as ancestry information) that have been made based on one or more shared IBD segments.

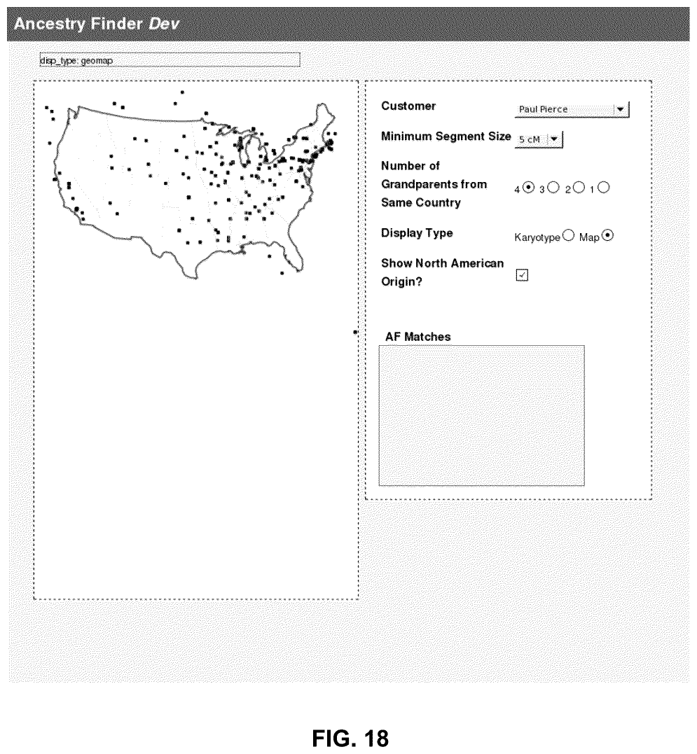

FIG. 18 shows an interface example for a geographical map view of an ancestry finder system. The left hand side shows a map of some region of the world. The dots are latitude/longitude coordinates inferred from text typed in by users describing their specific places of birth, and/or those of their ancestors. The dots are the birthplaces of US-born grandparents of other users who have an IBD match with user Paul Pierce. The match is based on a minimum segment size of 5 cM, minimum number of grandparents from the same country of 4, and North American origin. For example, the map could be of North America.

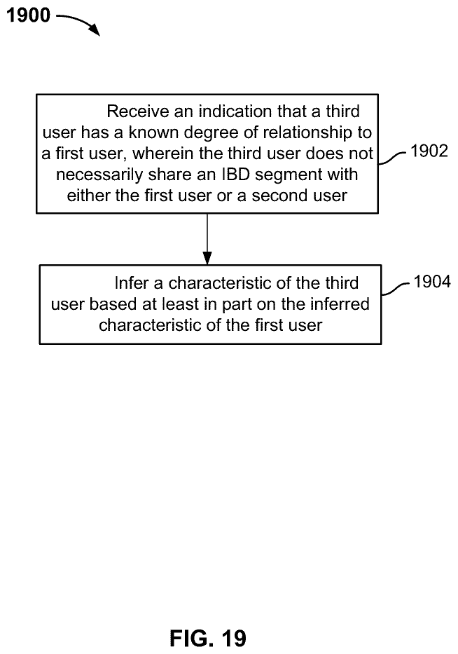

FIG. 19 is a flowchart illustrating an embodiment of a process for inferring a characteristic of an individual from the ancestry or other phenotypic information of a second individual, even if the two individuals are not related by DNA. Process 1900 may be implemented on an ancestry finder system such as 1100. The process may be invoked, for example, at a user's request to look for ancestry information. In some embodiments, this process is performed between 1106 and 1108 of process 1100, for cases in which a third user is requesting ancestry information. In this case, it is known that the third user is related to the first user, but the third user does not share an IBD segment with the second user. In some cases, the third user does not share an IBD segment with the first user either.

For example, the third user may be a sibling of the first user, but the third user does not have an IBD match with any other user in the database. However, the first user has an IBD match with the second user. Because it is known that the first user and the third user are siblings, and that the first user and second user have shared IBD for at least one segment, then, in some embodiments, ancestry information for the third user can be inferred from the ancestry information about the second user. In some cases, this means that it need not be determined whether the third user has any shared IBD segments with the second user, and this ancestry information can be inferred as soon as it is known that the third user is a sibling of the first user.

As a more specific example, the first user and the third user are full siblings, the second user is the father of the first user, the first user has a segment that is a shared IBD segment with his father (the second user), but the third user does not share that IBD segment with his father (the second user). If both of the father's parents are from Germany, then it can be inferred that the third user has at least two grandparents, his paternal grandparents, from Germany. If instead the relationship between the first user and second user is not known, perhaps because the second user is a distant cousin of the first user instead of the father, then it can at least be inferred that the third user has German ancestry, because the third user's lineages are identical to those of the first user's.

At 1902, an indication that a third user has a known degree of relationship to the first user is received. The third user does not necessarily share an IBD segment with either the first user or the second user. In the example above, the known degree of relationship between the third user and the first user is “sibling.”

At 1904, a characteristic of the third user is inferred based at least in part on the inferred characteristic of the first user. In the example above, it is inferred that the third user has German ancestry, or if it is known that the second user is the father of the first user, then it is inferred that the third user has at least two grandparents from Germany.

Although the foregoing embodiments have been described in some detail for purposes of clarity of understanding, the invention is not limited to the details provided. There are many alternative ways of implementing the invention. The disclosed embodiments are illustrative and not restrictive.

What is claimed is:

1. A system comprising:
two or more processors;
persistent memory containing program instructions that, when executed by the two or more processors cause the system to perform operations including:
identifying homozygous markers of a first user;
encoding, in a plurality of bitwise arrays, respective zygosity states of the markers for a plurality of other individuals, wherein lengths of the bitwise arrays match a word length of the two or more processors;
identifying, in parallel by the two or more processors and using bitwise operations on the plurality of bitwise arrays, identical-by-descent segments each of at least a minimum length that the first user shares with a subset of individuals from the plurality of other individuals;
obtaining, from one or more databases, respective ancestor birthplace information of the subset of individuals;
based on the respective ancestor birthplace information of the subset of individuals, estimating respective ancestor origins of each of the identical-by-descent segments to be countries, geographical regions, or ethnicities; and
causing to display, on a graphical user interface, the identical-by-descent segments arranged within their respective chromosomes and colored to indicate their respective ancestor origins.

2. The system of claim 1, wherein the respective ancestor birthplace information indicates that at least some of the subset of individuals have two or more grandparents who were born in a country, born in a geographical region, or had an ethnicity indicating the respective ancestor origins.

3. The system of claim 1, wherein the identical-by-descent segments arranged within their respective chromosomes are presented in a karyotype view.

4. The system of claim 1, wherein the identical-by-descent segments are identified based on their containing common markers.

5. The system of claim 1, wherein the identical-by-descent segments are bounded by opposite homozygous calls.

6. The system of claim 1, wherein an ancestor origin of at least one of the identical-by-descent segments comprises two or more different countries, geographical regions, or ethnicities.

7. The system of claim 1, wherein the first user and at least one of the subset of individuals were previously unknown to be related before the sharing of the identical-by-descent segments was determined.

8. The system of claim 1, wherein the respective ancestor origins are also displayed on the graphical user interface as a table of countries.

9. The system of claim 1, wherein the respective ancestor origins are also displayed on the graphical user interface as a pie chart of countries.

10. The system of claim 1, wherein the respective ancestor origins are also displayed on the graphical user interface as a map of countries.

11. The system of claim 1, the operations further comprising:
receiving an indication that a second user has a known degree of familial relationship to the first user; and
determining, based on the respective ancestor origins estimated for the identical-by-descent segments of the first user, an ancestor origin of the second user.

12. A computer-implemented method comprising:
identifying homozygous markers of a first user;
encoding, in a plurality of bitwise arrays, respective zygosity states of the markers for a plurality of other individuals, wherein lengths of the bitwise arrays match a word length of two or more processors;
identifying, in parallel by the two or more processors and using bitwise operations on the plurality of bitwise arrays, identical-by-descent segments each of at least a minimum length that the first user shares with a subset of individuals from the plurality of other individuals;
obtaining, from one or more databases, respective ancestor birthplace information of the subset of individuals;
based on the respective ancestor birthplace information of the subset of individuals, estimating respective ancestor origins of each of the identical-by-descent segments to be countries, geographical regions, or ethnicities; and
causing to display, on a graphical user interface, the identical-by-descent segments arranged within their respective chromosomes and colored to indicate their respective ancestor origins.

13. The computer-implemented method of claim 12, wherein the respective ancestor birthplace information indicates that at least some of the subset of individuals have two or more grandparents who were born in a country, born in a geographical region, or had an ethnicity indicating the respective ancestor origins.

14. The computer-implemented method of claim 12, wherein the identical-by-descent segments are identified based on their containing common markers.

15. The computer-implemented method of claim 12, wherein the identical-by-descent segments are bounded by opposite homozygous calls.

16. The computer-implemented method of claim 12, wherein an ancestor origin of at least one of the identical-by-descent segments comprises two or more different countries, geographical regions, or ethnicities.

17. The computer-implemented method of claim 12, wherein the first user and at least one of the subset of individuals were previously unknown to be related before the sharing of the identical-by-descent segments was determined.

18. The computer-implemented method of claim 12, wherein the respective ancestor origins are also displayed on the graphical user interface as a table, pie chart, or map of countries.

19. The computer-implemented method of claim 12, further comprising:
receiving an indication that a second user has a known degree of familial relationship to the first user; and
determining, based on the respective ancestor origins estimated for the identical-by-descent segments of the first user, an ancestor origin of the second user.

20. A system comprising:
two or more processors;
persistent memory containing program instructions that, when executed by the two or more processors cause the system to perform operations including:
determining, in parallel by the two or more processors and using bitwise operations on respective zygosity states of markers, that a first user shares identical-by-descent segments each of at least a minimum length with a plurality of other individuals;
obtaining, from one or more databases, respective ancestor birthplace information of the other individuals;
based on the respective ancestor birthplace information of the other individuals, estimating respective ancestor origins of each of the identical-by-descent segments to be countries, geographical regions, or ethnicities; and
causing to display, in a graphical user interface, the identical-by-descent segments colored to indicate their respective ancestor origins.
